SEQUENCE LISTING (4 sequences)

SEQ ID NO: 1 - DNA, 30 bp, synthetic construct
  misc_feature 1..30: note = Synthetic construct
  misc_feature 30: note = amino linker
  source 1..30: mol_type = other DNA
gatctgacagtaggtacgcatcaggacatc

SEQ ID NO: 2 - DNA, 30 bp, synthetic construct
  misc_feature 1..30: note = Synthetic construct
  misc_feature 1: note = amino linker
  source 1..30: mol_type = other DNA
gatacaggctgcaccattagcgacgggatc

SEQ ID NO: 3 - DNA, 30 bp, synthetic construct
  misc_feature 1..30: note = Synthetic construct
  source 1..30: mol_type = other DNA
gatgtcctgatgcgtacctactgtcagatc

SEQ ID NO: 4 - DNA, 30 bp, synthetic construct
  misc_feature 1..30: note = Synthetic construct
  source 1..30: mol_type = other DNA
gatcccgtcgctaatggtgcagcctgtatc

(19) United States
(12) Patent Application Publication
(10) Pub. No.: US 2024/0052401 A1
(43) Pub. Date: Feb. 15, 2024

(54) NANOTRAIN FOR SINGLE-MOLECULE DETECTION

(71) Applicant: Universal Sequencing Technology Corporation, Carlsbad, CA (US)

(72) Inventors: Peiming ZHANG, Carlsbad, CA (US); Xinyue ZHANG, Carlsbad, CA (US); Ming LEI, Sharon, MA (US)

(73) Assignee: Universal Sequencing Technology Corporation, Carlsbad, CA (US)

(21) Appl. No.: 18/492,566

(22) Filed: Oct. 23, 2023

Related U.S. Application Data
(63) Continuation of application No. PCT/US2022/026229, filed on Apr. 25, 2022.
(60) Provisional application No. 63/179,186, filed on Apr. 24, 2021.

(51) Int. Cl. C12Q 1/6825; G01N 33/487

(52) U.S. Cl. CPC C12Q 1/6825; G01N 33/48721

(57) ABSTRACT

Provided herein, in one aspect, is an improved nanotrain for use in connection with a nanopore device for single-molecule detection. Methods for making and using the same are also provided.

### CROSS-REFERENCE TO RELATED APPLICATIONS

This application is a continuation under 35 U.S.C. § 111(a) of PCT International Patent Application No. PCT/US2022/026229, filed Apr. 25, 2022, designating the United States and published in English, which claims priority to and the benefit of U.S. Provisional Application No. 63/179,186, filed Apr. 24, 2021, the entire contents of each of which are incorporated by reference herein.

### SEQUENCE LISTING

The present application contains a Sequence Listing which has been submitted electronically in XML format following conversion from the originally filed TXT format. The content of the electronic XML Sequence Listing, (Date of creation: Oct. 22, 2023; Size: 6,709 bytes; Name: 168460-012302US-Sequence_Listing.xml), is herein incorporated by reference in its entirety.

### FIELD

This disclosure relates to a one-dimensional water-soluble nanoarray for single-molecule detection of analytes, herein referred to as a nanotrain, and methods for making the same.

### BACKGROUND

Most disease states involve or disrupt multiple biochemical pathways, and often the quantification of various biomarkers is necessary to diagnose and understand a disease comprehensively. For example, a patient's miRNA profile can be a diagnostic signature, which requires the quantification of multiple miRNAs.¹ Also, simultaneous measurement of different protein molecules is critically vital for the discovery of biomarkers², the early detection of cancer, disease monitoring, and personalized cancer therapy. Detection of panels of protein markers can minimize false positives and negatives in cancer diagnoses that could arise from measuring a single biomarker. During the COVID-19 pandemic, the multiplexed test has been employed to assess different anti-SARS-CoV-2 antibodies for enhanced clinical sensitivity.^{3, 4}

The well-known technologies for multiplex detection are microarrays⁵⁻⁸ and microbeads⁹⁻¹² with molecular probes attached to solid surfaces; however, the signal readout has mainly relied on optical or electrochemical methods at an ensemble-average level. A water-soluble 2D nanoarray has been developed using the DNA self-assembly to detect RNA detection^{13, 14} and protein¹⁵, which used atomic force microscopy (AFM) to examine the binding events. However, as a single molecule technique, AFM requires a well-trained person to conduct measurements and interpret data. Also, the cost and time of an AFM test may be another factor to limit its use in clinical diagnostics.

In the past two decades, nanopore technology has been developed for nucleic acid sequencing and molecular detection. A nanopore is an orifice of nanometer diameters and depths assembled from biological or fabricated from inorganic materials.¹⁶ It can function as a nanofluidic channel for the flow of ions and the transport of biomolecules. When a charged molecule is driven to pass through the nanopore electrophoretically, it modulates the ionic current by partially obstructing ionic flow. As a result, the current blockade signals are recorded and are used to identify the molecule and even its structural subunits. The nanopore is a single molecule sensor for detecting DNA¹⁷, RNA¹⁸⁻²⁰, proteins^{21, 22}, polysaccharides²³⁻²⁶, and other molecules. DNA can effectively translate through the nanopore due to the charges uniformly distributed along its phosphate backbone under physiological conditions. However, many molecules carry no charges, or zero in sum, which would make the electrophoretic translocation inefficient. Thus, improved carriers are needed in connection with the nanopore technology.

### BRIEF DESCRIPTION OF THE DRAWINGS

FIG. 1 illustrates an exemplary DNA nanotrain carrying different molecular cargoes, which causes fluctuations of an ionic current as it is translocated through a nanopore device.

FIGS. 2A-2D illustrate an exemplary process of loading cargoes to a DNA nanotrain.

FIG. 3: an exemplary route to synthesizing a DNA nanotrain head and its attachment to microbeads for assembling a DNA nanotrain.

FIGS. 4A-4B: (FIG. 4A) an exemplary route to synthesizing DNA carriage components; (FIG. 4B) an exemplary route to synthesizing a poly(ethylene glycol) linker for assembling a DNA nanotrain.

FIG. 5 shows an exemplary synthetic cycle for synthesizing a DNA nanotrain on a solid support.

FIG. 6 illustrates exemplary modified DNA structures for loading affinity molecules to the carriages of a nanotrain.

FIG. 7: an exemplary route to attaching an aptamer to the modified DNA containing an amino-modified thymidine.

FIGS. 8A-8B: (FIG. 8A) a route to modifying a DNA carriage with azido functionalized poly(ethylene glycol)₃₆ (PEG₃₆) at its 3′-end; (FIG. 8B) a route to modifying a DNA carriage with DBCO functionalized PEG₂₄ at its 5′-end.

FIGS. 9A-9B: (FIG. 9A) an exemplary route to preparing a nanotrain of two DNA carriages connected via a PEG₆₀ linker; (FIG. 9B) a nanotrain of two DNA carriages connected via a PEG₂₄ linker.

FIG. 10: a gel electrophoresis image of DNA nanotrains and their components.

FIG. 11: a gel electrophoresis image of DNA nanotrains hybridizing with their complementary moieties.

### SUMMARY

In one aspect, a nanotrain is provided, comprising:
a plurality of single-stranded DNA carriages arranged linearly, each having a unique sequence;
a plurality of complementary DNA sequences each predesigned to be complementary to a single-stranded DNA carriage, and each hybridized with its complementary single-stranded DNA carriage;
a plurality of affinity molecules for capturing one or more targets, wherein each affinity molecule is constructed to attach to a complementary DNA sequence; and
a plurality of flexible linkers connecting every two adjacent single-stranded DNA carriages, wherein each flexible linker at a first end is connected to 5′-end of a first single-stranded DNA carriage and at a second end is connected to 3′-end of a second single-stranded DNA carriage.

In some embodiments, each single-stranded DNA carriage and each complementary DNA sequence is independently selected from xeno nucleic acid (XNA), peptide nucleic acids (PNA), locked nucleic acid (LNA), and cyclohexenyl nucleic acids (CeNA), wherein preferably each single-stranded DNA carriage and each complementary DNA sequence has a length ranging from 6 to 1000 bases.

In some embodiments, each complementary DNA sequence is modified to have a functional group that is predesigned to attach an affinity molecule thereto, wherein preferably the functional group is selected from amine, thiol, azide, alkyne, cycloalkyne, or tetrazine.

In some embodiments, each affinity molecule is independently selected from one or more of nucleic acid, XNA, aptamer, ligand, antibody, antibody's fragment, antigen, nanobody, affibody, protein, and/or carbohydrate.

In some embodiments, each affinity molecule comprises a microparticle such as a magnetic bead, amine, thiol, azide, alkyne, cycloalkyne, and/or tetrazine.

In some embodiments, the one or more targets are selected from multiplexed protein markers, single nucleotide polymorphisms (SNPs), DNA and RNA mutations, structural variations of a genome, drug molecules, antibodies, antigens, and glycans.

In some embodiments, each single-stranded DNA carriage further comprises a protein molecule carrying at least one function orthogonal to those occurring in natural amino acids, such as oxyamine, hydrazine, aldehyde, azide, alkyne, cycloalkyne, alkene, or tetrazine.

In some embodiments, each single-stranded DNA carriage further comprises a charged polysaccharide that carries functional groups such as thiol, azide, alkyne, cycloalkyne, tetrazine, or oxyamine.

In some embodiments, each flexible linker comprises a polypeptide having the following structure:

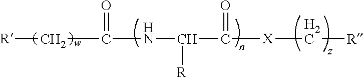

where n=2 to 200; w=1 to 10; z=0 to 10
X=0, NH, or ONH;
R═H, amine, thiol, azide, alkyne, cycloalkyne, tetrazine, carboxylate, hydroxyl, alkyl, alkene, guanidinium, or glycan, selenium;
R′=azide, alkyne, cycloalkyne, tetrazine, or aldehyde;
R″═H, azide, alkyne, cycloalkyne, cyclooctene, tetrazine, or aldehyde.

In some embodiments, each flexible linker comprises a poly(ethylene glycol) having the following structure:

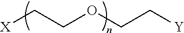

where n=2 to 500;
X=amine, maleimide, vinyl sulfone, cyclooctene, thiol, azide, alkyne, cycloalkyne, tetrazine, oxyamine, carboxylate, or aldehyde;
Y=amine, maleimide, vinyl sulfone, thiol, azide, alkyne, cycloalkyne, tetrazine, oxyamine, carboxylate, or aldehyde.

In some embodiments, the nanotrain can further include a neutral tail.

In some embodiments, the nanotrain can further include a magnetic bead.

Another aspect relates to a system for single-molecule detection, comprising:
any one of the nanotrain disclosed herein,
a nanopore through which the nanotrain translocates, wherein the nanopore is formed by a biological, organic, inorganic, natural or synthetic material and has a pore diameter and thickness in a range of 2 to 1000 nm, preferably 2 to 50 nm; wherein optionally a first pair of electrodes is embedded within the nanopore for measuring current, voltage and/or capacity;
a nanopore device with a cis reservoir and a trans reservoir that are separated by a membrane with the nanopore embedded therein;
a bias voltage that is applied between the cis and trans reservoirs through a second pair of electrodes;
a device for recording a current, voltage or capacity fluctuation caused by the nanotrain translocating through the nanopore; wherein the current, voltage or capacity fluctuation detects one or more targets captured by the affinity molecules; and
software for data analysis that identifies or characterizes the one or more targets.

In some embodiments, the one or more targets are selected from multiplexed protein markers, single nucleotide polymorphisms (SNPs), DNA and RNA mutations, structural variations of a genome, drug molecules, antibodies, antigens, and glycans.

In some embodiments, the system can include a plurality of nanotrains, wherein the nanopore device comprises a plurality of nanopores, preferably an array of nanopores, wherein preferably the nanopore device contains 10 to 10⁹ nanopores, preferably 10³ to 10⁷ nanopores, or more preferably 10⁴ to 10⁶ nanopores.

A further aspect relates to a method for single-molecule detection, comprising:
a. providing the system disclosed herein;
b. mixing the nanotrain with a sample containing one or more targets, thereby forming a loaded nanotrain having the one or more targets captured thereto via the plurality of affinity molecules;
c. optionally separating the loaded nanotrain from the sample, preferably via the magnetic bead which can be removed after the separating step;
d. placing the loaded nanotrain into the nanopore device, preferably in the cis reservoir;
e. applying the bias voltage between the cis and trans reservoirs to translocate the loaded nanotrain through the nanopore; and
f. recording a current, voltage or capacity fluctuation caused by the loaded nanotrain translocating through the nanopore; wherein the current, voltage or capacity fluctuation detects the one or more targets captured by the affinity molecules.

In some embodiments, the method is for high-throughput detection of a plurality of targets using a nanopore device comprising a plurality of nanopores, preferably an array of nanopores, wherein preferably the nanopore device contains 10 to 10⁹ nanopores, preferably 10³ to 10⁷ nanopores, or more preferably 10⁴ to 10⁶ nanopores.

A method of synthesizing a nanotrain is also provided, comprising:
a. providing a plurality of carriages and flexible linkers, wherein each carriage is a single-stranded DNA carriages having a unique sequence, wherein each carriage has orthogonal functional groups attached to its 5′-end and 3′-end, wherein each functional group is independently selected from an amine, maleimide, thiol, vinyl sulfone, azide, alkyne, cycloalkyne, cyclooctene, or oxyamine;
b. attaching a head carriage via a cleavable linker to a solid support, forming a first end of a nanotrain;
c. attaching a first flexible linker to the head carriage;
d. attaching a first carriage to the first flexible linker;
e. attaching a Nth flexible linker to the (N−1)th carriage, wherein N is an integer ≥2;
f. attaching a Nth carriage to the Nth flexible linker;
g. attaching a (N+1)th flexible linker to the Nth carriage;
h. repeating steps (e)-(g) until a desired number of carriages are linked by the flexible linkers;
i. attaching a neutral tail to a second end of the nanotrain; and
j. cleaving the nanotrain from the solid support at the cleavable linker.

### DETAILED DESCRIPTION

Provided herein, in some embodiments, is a negatively charged DNA molecule used as a carrier for the nanopore-based detection of proteins. A protein molecule captured on a double-stranded DNA can be detected by translocation through a nanopore.^{27, 28}

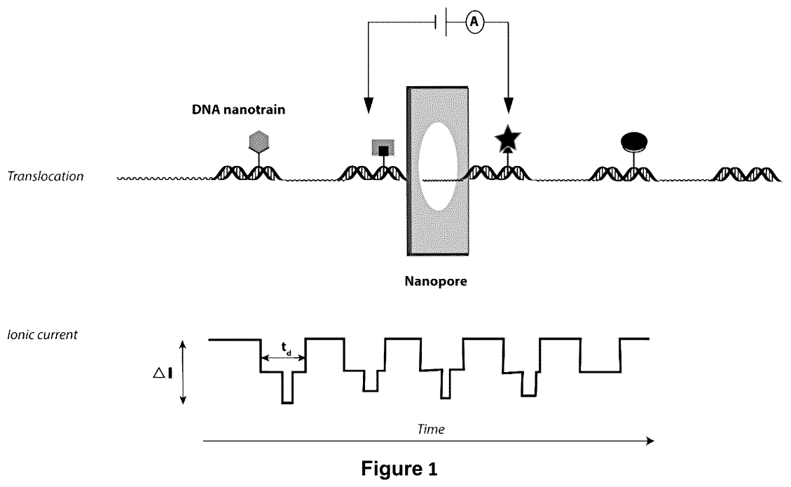

This disclosure provides a one-dimensional nanoarray, referred to as a nanotrain, for sensing biomolecules using a nanopore apparatus, wherein the nanopores are either biological, synthetical, solid-state, or the combination thereof, and the nanopore size is about 2 nm to 1000 nm, preferably 2 nm to 50 nm. FIG. 1 illustrates a DNA nanotrain carrying multiplex molecular cargoes sensed by a nanopore. When the nanotrain is electrophoretically translocated through the nanopore, each of its cargoes blocks electrolytes from flowing, causing fluctuation of the ionic current. The change in ionic current is measured and characterized by its magnitude (AI) and duration (td). These resistive pulse parameters and the peak position can be used to identify individual payloads. Such a nanotrain can be used to detect multiplexed protein markers, single nucleotide polymorphisms (SNPs), DNA and RNA mutations, structural variations of a genome, drug molecules, antibodies, antigens, and glycans.

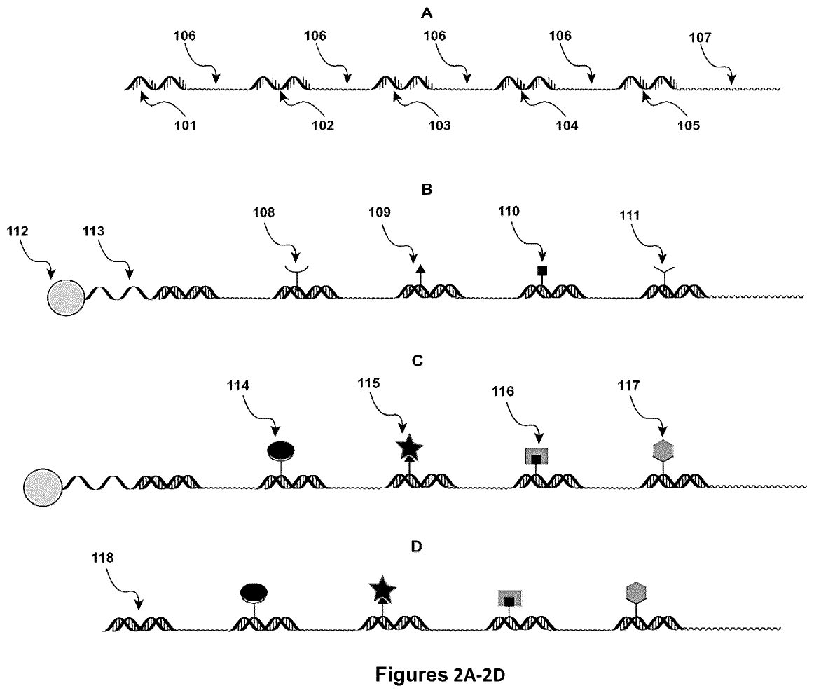

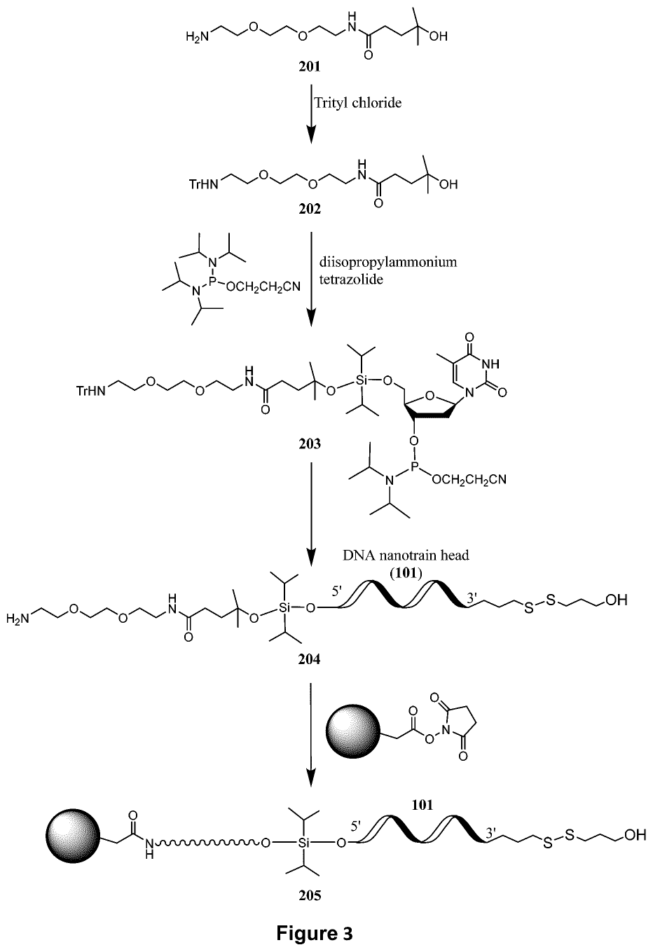

FIG. 2A shows a typical DNA nanotrain comprising a train head (single-stranded DNA, 101) and four consecutive DNA carriages (single-stranded DNA, 102, 103, 104, 105) were connected to one another through a flexible linker (106) with a neutral charge tail (107) at its end. These DNA carriages can be distinguished from one another by their sequences. For detection, for example, of proteins, the nanotrain is equipped with receptors conjugated to complementary DNA (108, 109, 110, 111) and connected to a magnetic bead (112) functionalized with a single-stranded DNA (113) by hybridization (FIG. 2B). 113 can be made in the same way as making 205 in FIG. 3. The sequence of 113 is complementary to 101. 113 can be DNA or its analogies, such as LNA, cyclohexenyl nucleic acids (CeNA). When the equipped nanotrain is mixed with a sample solution, it captures those cognate ligands (114, 115, 116, 117, FIG. 2C). The nanotrain loaded with cargoes can be separated from the solution by fixing it on a vial wall through a magnetic force and then released by cutting the single-stranded DNA fragment next to the magnetic bead using a nuclease. Thus, a loaded nanotrain with a double-stranded DNA head (118, FIG. 2D) is ready for a nanopore analysis as described in the above section.

In some embodiments, poly(ethylene glycol) (PEG) can be used as a linker to connect DNA carriages to form a nanotrain. Compared with X DNA carrier²⁸, or double-stranded DNA²⁹, the neutral and flexible PEG linker can facilitate the entry of the nanotrain into a nanopore, increasing the capture rate of the nanopore. It also extends the dwell time of the nanotrain in the nanopore, resulting in increased electrical signals. PEG has been studied as a crowding molecule, which increases the event frequency and dwell time.³⁰

The embodiments described and claimed herein have many attributes and embodiments, including, but not limited to, those set forth or described or referenced in this disclosure. The embodiments described and claimed herein are not limited to, or by, the features or embodiments identified in this disclosure, which is for illustration purposes only but not for restriction. The following will use DNA nanotrain as an example.

### Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, the term “nanotrain” refers to a linear molecular structure comprising a series of recognition entities that are concatenated by a linkage molecule. These recognition entities act as train carriages to carry cargoes (analytes). The sectional backbone of the train carriage can be made of DNA, RNA, peptide nucleic acid (PNA), polypeptides, polysaccharides, antibodies, receptors, supramolecules, and any linear molecule, charged or non-charged, either natural, modified, or synthesized or the combination thereof. A part or whole of a backbone section can be the recognition entity or be topped or conjugated with a recognition entity, such as a probe, receptor, antibody, molecular tweezer, etc. Specifically, if the carriage comprises DNA, it is called a DNA nanotrain. The linkage molecule is an essential part of the nanotrain that is designed to cause distinct signals from that of the train carriage's cargo section, usually with different sizes (diameters) or other properties that attribute to distinct nanopore signals.

Various aspects of the disclosure are described in further detail below. Additional definitions are set out throughout the specification.

In one embodiment, provided herein a route to synthesizing the DNA nanotrain illustrated in FIG. 2A. At first, a precursor of the DNA train head (101) is synthesized following a process described in FIG. 3. The amino group of N-(2-2-(2-aminoethoxy)ethoxy) ethyl)-4-hydroxy-4-methylpentanamide (201) first reacts with trityl chloride (TrCl), giving an amino protected product (202) The compound is introduced to the 5′-end of a nucleoside as a traceless cleavable linker using Bergstrom's method³¹. For example, compound 202 is attached to the 5′-end of thymidine by reacting with diisopropyldichlorsilane in the presence of diisopropylethylamine and imidazole with the nucleoside in DMF. The resulting compound is subjected to phosphinylation, giving a thymidine phosphoramidite containing a cleavable linker (203). The phosphoramidite (203) is incorporated into the 5′-end of the DNA nanotrain head (101) to form the precursor (204) by an automated DNA synthesis. The nanotrain head is attached to a microbead functionalized with an N-hydroxvsuccinitnide (NHS) ester through its amino group (205, FIG. 3). The nanotrain head precursor also carries a disulfide linker to connect a DNA carriage at its 3′-end for assembling a DNA nanotrain.

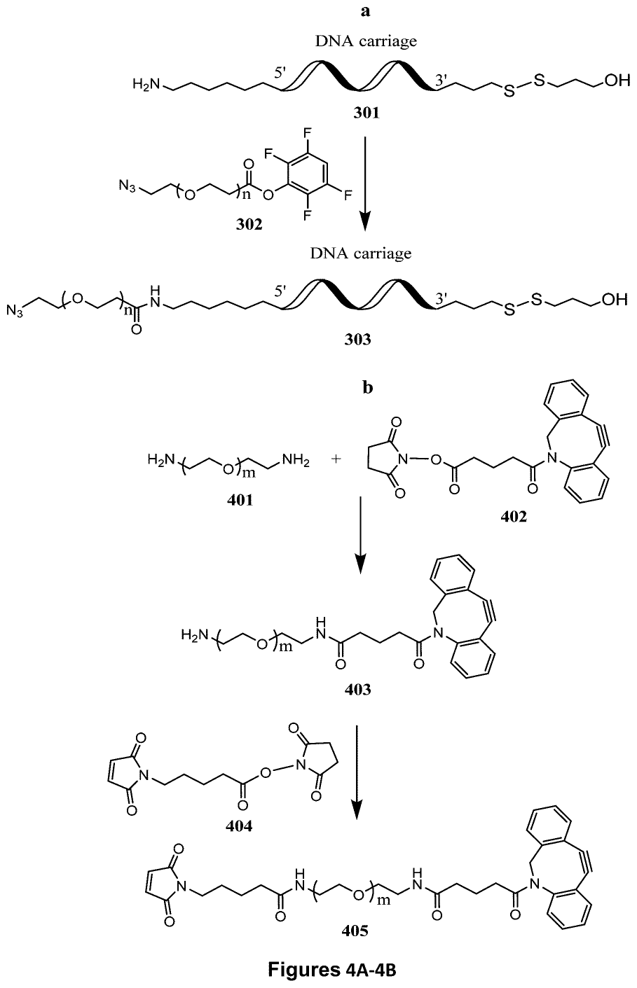

In some embodiments, methods for preparing nanotrain DNA components for assembling the DNA nanotrain are provided. FIG. 4A delineates a route to synthesizing a DNA carriage, such as 102, 103, 104, and 105 described in FIG. 2A, which bears a PEG linker with an azido function at its 5′-end and a disulfide at its 3′-end (303). In detail, a DNA carriage containing an alkylamine at its 5′-end and a disulfide function at its 3′-end (301) are synthesized in a conventional automated DNA synthesizer. DNA 301 can readily be converted to azide functionalized DNA 303 by reacting with the azido-dPEGn-TFP ester (302). Similarly, FIG. 4B shows a route to synthesizing a PEG linker to connect DNA carriages in the DNA nanotrain. Diamino-PEG (401) first reacts with Dibenz[b,f]azocine-5(6H)-pentanoic acid, 11,12-didehydro-6-oxo-, 2,5-dioxo-1-pyrrolidinyl ester (DBCO-C5-NHS-ester, 402), yielding an amino-PEG linker containing a DBCO function at its one end (403), It further reacts with 5-maleimidovalericacid-NHS ester (404), generating a PEG linker containing a maleimide function at its other end (405). These two functional groups of the PEG linker allow for assembling a DNA nanotrain through orthogonal click chemistry.

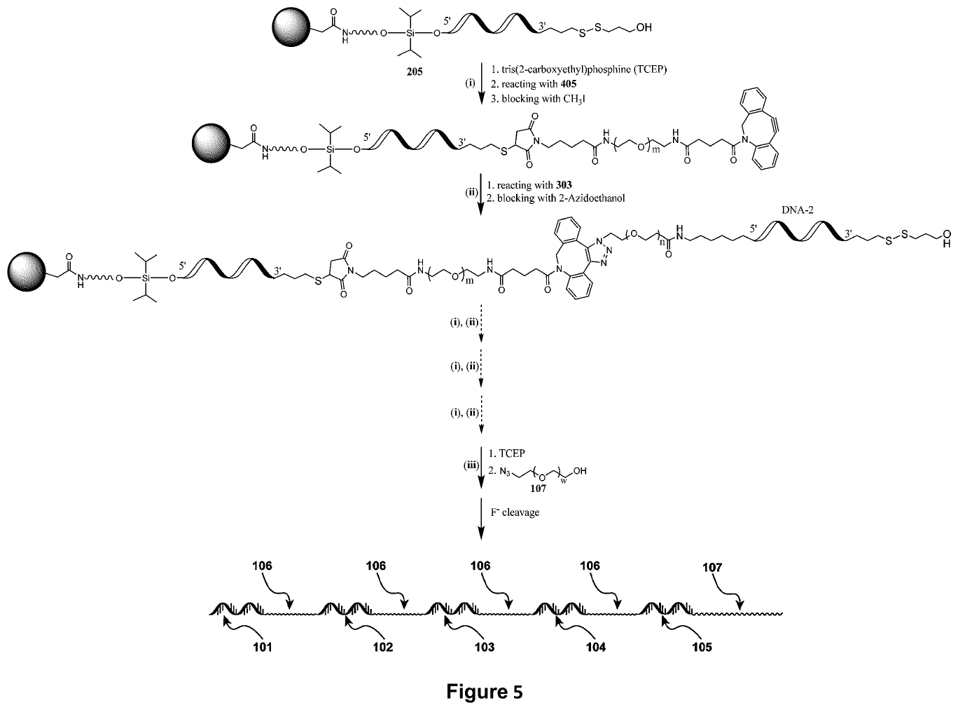

In one embodiment, a synthetic cycle for assembling a nanotrain on a microbead is provided (FIG. 5). The synthesis starts from the nanotrain head precursor attached to a microbead (205). It is first treated with TCEP to reduce the disulfide to thiol, followed by reacting with the PEG linker 405 (Step i) and blocking the unreacted thiol with iodomethane (CH₃I). Next, the microbead is allowed to react with component 303 containing a DNA carriage 102 and the blocking reagent 2-azidoethanol (Step ii) to finish the first synthesis cycle. By repeating Steps i and ii, the DNA carriage 103, 104, and 105 are connected to the DNA nanotrain, respectively. The last step (iii) is connecting an azido-PEG tail (501) to the DNA nanotrain to complete the whole process. Then, the product is cleaved from the microbead using a fluoride reagent reported in literature³¹.

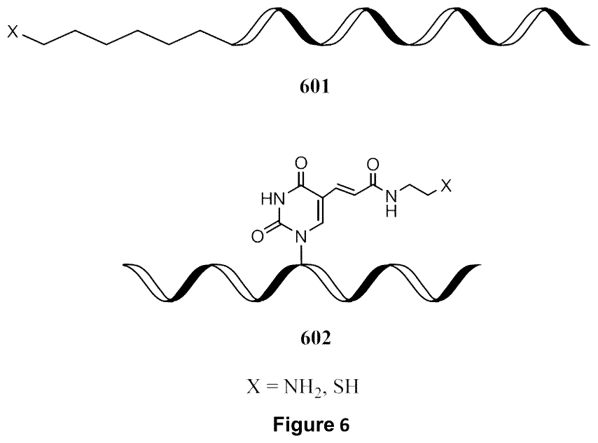

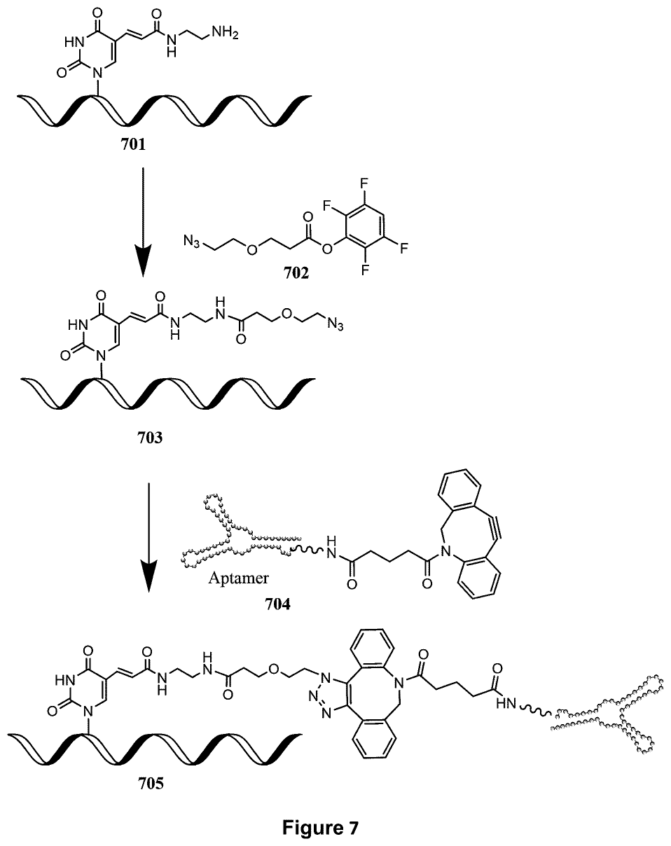

In some embodiments, methods for loading different affinity molecules to the DNA carriages for detecting the targeting analytes are provided. First, a set of DNA molecules with sequences complementary to those of the carriages in the nanotrain is synthesized, respectively, to contain a function group, such as amine, thiol, or others (FIG. 6). The function group is placed either at the end of the complementary DNA (601) or on the internal DNA base (602). These functional groups are employed for attaching the affinity molecules to DNA. For example, FIG. 7 delineates a route to attaching an aptamer to DNA. First, complementary DNA to a carriage is synthesized to contain an internal amino-modified thymidine (701). Then, the DNA reacts with 3-(2-azidoethoxy)propanoic acid-2,3,5,6-tetrafluorophenyl ester (702) to yield an azido functionalized DNA (703). An aptamer functionalized with amine reacts with DBCO-C5-NHS-ester (402) to generate an aptamer containing a DBCO function (704). Mixing DNA 703 with aptamer 704 allows a click reaction to occur spontaneously, giving a DNA-aptamer conjugate (705). The conjugate is loaded to the DNA nanotrain by hybridizing to the DNA carriage with a complementary sequence.

In some embodiments, different analytes are sensed/detected separately or in a combination of two or more kinds of analytes, wherein the analytes include but are not limited to DNA, RNA, proteins, antibodies/antigens, saccharides, any biological analytes, organic analytes, etc.

In some embodiments, a plurality of nanotrain molecular structures is constructed and used with a nanopore chip device containing a plurality of nanopores or an array of nanopores. In some embodiments, the nanopore array contains 10 to 10⁹ nanopores, preferably 10³ to 10⁷ nanopores, or more preferably 10⁴ to 10⁶ nanopores.

In some embodiments, the nanopore chip device is designed and constructed in the nanopore chip device as described in, WO201707562, WO2018209286, and PCT/US20/042188, all incorporated herein by reference. The nanopore can be any shape such as those described therein. The nanotrain and its respective targets can be measured using the ionic current blockage method or using electrodes embedded in the nanopore such as those described therein.

### EXAMPLES

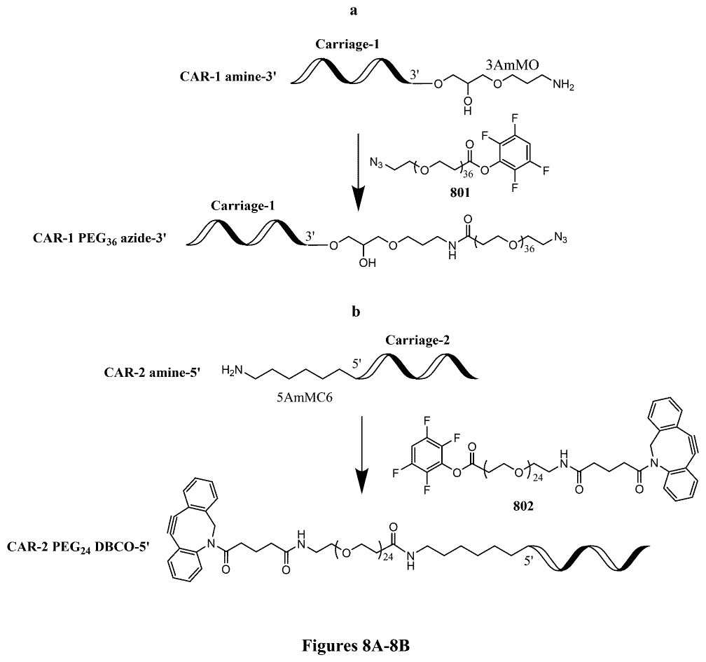

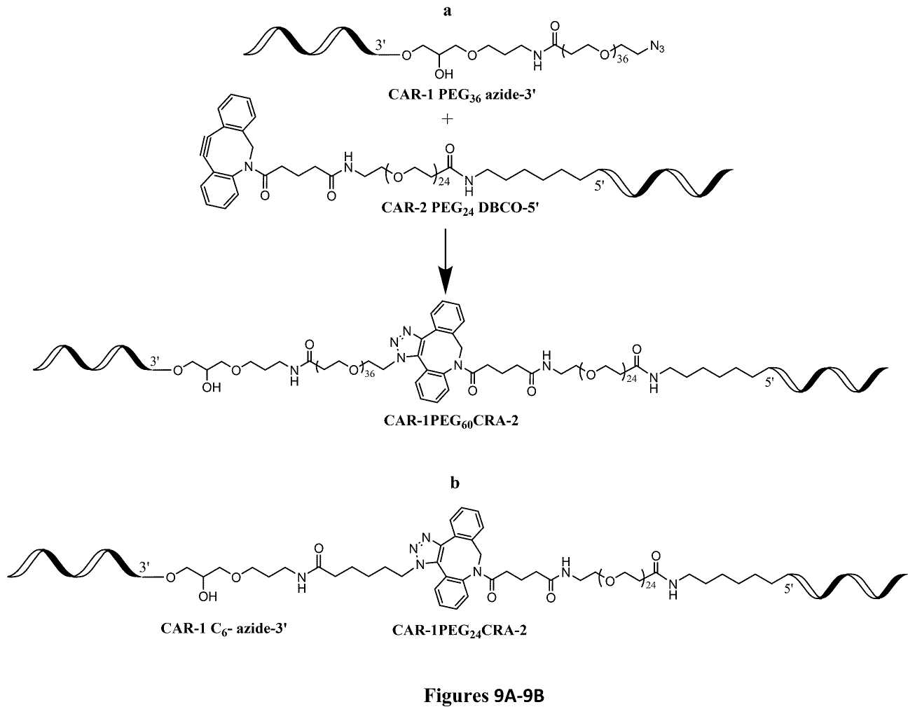

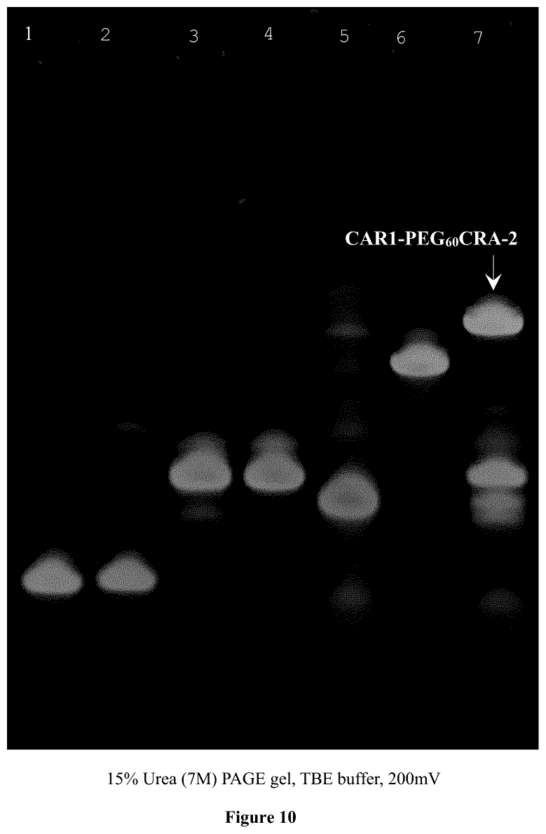

In one embodiment, a nanotrain containing two DNA carriages can be prepared. As an example, DNA carriage-1 has a sequence of 5′-GAT CTG ACA GTA GGT ACG CAT CAG GAC ATC/3AmMO/-3′ (SEQ ID NO. 1) with an amino linker (3AmMO) at 3′-end (CAR-1 amine-3′), and the carriage-2 has 5′-/5AmMC6/GAT ACA GGC TGC ACC ATT AGC GAC GGG ATC-3′ (SEQ ID NO. 2) with an amino linker (5AmMC6) at 5′-end (CAR-2amine-5′), shown in FIGS. 8A-8B. CAR-1amine-3′ reacted with Azido-dPEG ° 36-TFP ester (801), resulting in a DNA carriage containing a PEG linker with 36 ethylene glycol units long and ended an azide at its 3′-end (FIG. 8A, CAR-1PEG₃₆ azide-3′). Similarly, CAR-2amine-5′ reacted with DBCO-dPEG®₂₄-TFP ester (802), resulting in a DNA carriage containing a PEG linker with 24 ethylene glycol units long and ended a DBCO at its 5′-end (CAR-2PEG₂₄DBCO-5′, FIG. 8B). Finally, mixing CAR-1PEG₃₆ azide-3′ with CAR-2PEG₂₄DBCO-5′ in an aqueous solution produced a nanotrain with two DNA carriages connected via a PEG linker of 60 ethylene glycol units (CAR-1PEG₆₀CAR-2, FIG. 9-a). Similarly, a nanotrain with two DNA carriages connected via a PEG linker of 24 ethylene glycol units (CAR-1PEG₂₄CAR-2) was assembled by CAR-106-azide-3′ reacting with CAR-2PEG₂₄DBCO-5′ in the aqueous solution (FIG. 9-b). These desired products were verified by gel electrophoresis (FIG. 10). Lane 1: CAR-1 amine-3′; Lane 2: CAR-2 amine-5′; Lane 3: gel purified CAR-1 PEG₃₆ azide-3′ (sample #2); Lane 4: gel purified CAR-1 PEG₃₆ azide-3′ (sample #2); Lane 5: precipitated CAR-2 PEG₂₄ DBCO-5′; Lane 6: purified CAR-1PEG₂₄CAR-2; Lane 7: a reaction mixture for producing CAR-1PEG₆₀CAR-2.

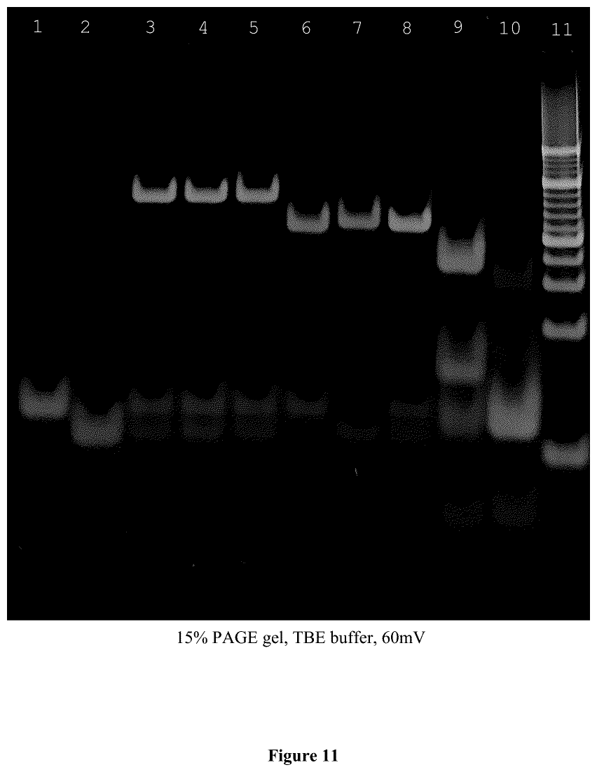

In some embodiments, the above said nanotrains are charged with complementary DNA moieties. Examples include two DNA entities, 5′-GAT GTC CTG ATG CGT ACC TAC TGT CAG ATC-3′ (CAR-1C) (SEQ ID NO. 3) and 5′-GAT CCC GTC GCT AAT GGT GCA GCC TGT ATC-3′ (CAR-2C) (SEQ ID NO. 4) made complementary to CAR-1 and CAR-2, respectively. The gel electrophoresis analysis indicates that CAR-1PEG₆₀CAR-2 and CAR-1PEG₂₄CAR-2 effectively hybridized with the complementary DNA entities, although they were assembled through PEG linkers with different lengths (FIG. 11). Lane 1: CAR-1C; Lane 2: CAR-2C; Lane 3: CAR-1PEG₆₀CAR-2+CAR-1C+CAR-2C (ratio=1: 1: 1); Lane 4: CAR-1PEG₆₀CAR-2+CAR-1C+CAR-2C (ratio=1: 1.1: 1.1); Lane 5: CAR-1PEG₆₀ CRA-2+CAR-1C+CAR-2C (ratio=1.1: 1: 1); Lane 6: CAR-1PEG₆₀CAR-2+CAR-1C (ratio=1: 1); Lane 7: CAR-1PEG₆₀ CRA-2+CAR-2C (ratio=1: 1); Lane 8: CAR-1PEG₂₄CAR-2+CAR-1C+CAR-2C (1: 1: 1); Lane 9: CAR-1PEG₆₀CAR-2 reaction mixture; Lane 10: CAR-2 PEG₂₄ DBCO-5′; Lane 11: O'Range Ruler 20 bp DNA ladder. Furthermore, those unhybridized residues can readily be separated from the products.

### Additional Embodiments

System for identifying and sensing single and multiplex molecules:
a. A nanopore that comprises a biological, organic, or inorganic material and has a pore diameter and thickness in a range of 2 to 1000 nm, preferably 2 to 50 nm;
b. A nanotrain that contains multiple carriages (n>1), each with an affinity molecule attached for the capture of their respective targets, wherein the carriages comprise a charged head and a neutral tail, and interspersed with a flexible linker, and wherein the affinity molecule includes nucleic acid, XNA, aptamer, ligand, antibody, antibody's fragment, antigen, nanobody, affibody, protein, or carbohydrate, but not limited to them, wherein the affinity molecule contains amine, thiol, azide, alkyne, cycloalkyne, or tetrazine, but not limited to them;
c. A nanopore device with two reservoirs that are separated by a membrane with a nanopore embedded;
d. A bias voltage that is applied between the said two reservoirs through a pair of electrodes;
e. A device that records a current fluctuation caused by the said nanotrain translocating the nanopore;
f. Software for data analysis that identifies or characterizes the target biomolecules
g. DNA carriages comprising nucleic acids, including DNA with a length ranging from 6 to 1000 bases, RNA with a length ranging from 6 to 1000 bases, which can form a duplex, triplex, quadruplex with itself or its complementary nucleic acid, wherein the nucleic acid and its complementary contain a functional group internally, or at the end, that can attach an affinity molecule to the carriage, including amine, thiol, azide, alkyne, cycloalkyne, or tetrazine, but not limited to them, and wherein the nucleic acid and its complementary comprise natural nucleosides, unnatural nucleosides, or their combinations;
h. DNA carriages with a xeno nucleic acid (XNA), including peptide nucleic acids (PNA), locked nucleic acid (LNA), cyclohexenyl nucleic acids (CeNA), but not limited to them, which can form a duplex, triplex, quadruplex with itself or its complementary XNA, wherein the XNA and complementary XNA contain a functional group internally, or at the end, that can attach an affinity molecule to the carriage, including amine, thiol, azide, alkyne, cycloalkyne, or tetrazine, but not limited to them, and wherein the XNA and complementary XNA comprise natural nucleosides, unnatural nucleosides, or their combinations;
i. The carriage comprising a protein molecule carrying at least one function orthogonal to those occurring in natural amino acids, which includes oxyamine, hydrazine, aldehyde, azide, alkyne, cycloalkyne, alkene, or tetrazine, but not limited to them
j. The carriage of the above Section b comprises a protein molecule carrying at least one function orthogonal to those occurring in natural amino acids, which includes oxyamine, hydrazine, aldehyde, azide, alkyne, cycloalkyne, alkene, or tetrazine, but not limited to them;
k. The carriage of the above Section b comprises a charged polysaccharide that carries functional groups including thiol, azide, alkyne, cycloalkyne, tetrazine, or oxyamine, but not limited to them;
l. The flexible linker of the above Section b comprises a polypeptide

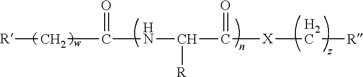

where n=2 to 200; w=1 to 10; z=0 to 10 X=0, NH, or ONH;
R═H, amine, thiol, azide, alkyne, cycloalkyne, tetrazine, carboxylate, hydroxyl, alkyl, alkene, guanidinium, or glycan, selenium, but not limited to them.
R′=azide, alkyne, cycloalkyne, tetrazine, or aldehyde, but not limited to them R″═H, azide, alkyne, cycloalkyne, cyclooctene, tetrazine, or aldehyde, but not limited to them;
m. The flexible linker of the above Section b comprising a poly(ethylene glycol) with a structure as described below:

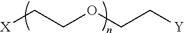

where n=2 to 500
X=amine, maleimide, vinyl sulfone, cyclooctene, thiol, azide, alkyne, cycloalkyne, tetrazine, oxyamine, carboxylate, or aldehyde, but not limited to them;
Y=amine, maleimide, vinyl sulfone, thiol, azide, alkyne, cycloalkyne, tetrazine, oxyamine, carboxylate, or aldehyde, but not limited to them;
n. The flexible linker of the above Section b comprise a combination of the polypeptide in the above Section m and poly(ethylene glycol) in the above Section 1.

Process of synthesizing a nanotrain on a solid support:
a. a cyclic method of synthesizing the nanotrain.
b. a cleavable linker at one end of the nanotrain head, through which the first component of the nanotrain is connected to the solid support.
c. orthogonal functional groups attached to two ends of a carriage, wherein the functional group is an amine, maleimide, thiol, vinyl sulfone, azide, alkyne, cycloalkyne, cyclooctene, oxyamine, but not limited to them
d. a synthesis cycle of repeating the incorporation of a flexible linker and a carriage component to the nanotrain head sequentially.
e. incorporation of a neutral tail into the end of the nanotrain.
f. cleavage of nanotrain from the solid support.
Wherein the solid support includes a microbead, nanobead, flat substrate, but is not limited to them; and wherein the solid support comprises an inorganic material, metal, metal oxide, polymer material, organic material, but are not limited to them.

All publications, patents, and other documents mentioned herein are incorporated by reference in their entirety. Unless defined otherwise, all technical and scientific terms used herein have the same meaning as those commonly understood by one of ordinary skill in the art to which this disclosure belongs. While the present disclosure has been illustrated by a description of various embodiments, and while these embodiments have been described in considerable detail, it is not the intention of the applicants to restrict or in any way limit the scope of the applications. Additional advantages and modifications will readily appear to those skilled in the art. Therefore, the disclosure in its broader aspects is not limited to the specific details, representative device, apparatus and method, and illustrative example shown and described. Accordingly, departures may be made from such details without departing from the spirit of the applicant's general inventive concept.

### CITATIONS

1. Jet, T.; Gines, G.; Rondelez, Y.; Taly, V., Advances in multiplexed techniques for the detection and quantification of microRNAs. Chemical Society Reviews 2021, 50, 4141-4161.
2. Serna, G.; Ruiz-Pace, F.; Cecchi, F.; Fasani, R.; Jimenez, J.; Thyparambil, S.; Landolfi, S.; Elez, E.; Vivancos, A.; Hembrough, T.; Tabernero, J.; Dienstmann, R.; Nuciforo, P., Targeted multiplex proteomics for molecular prescreening and biomarker discovery in metastatic colorectal cancer. Scientific reports 2019, 9, 13568.
3. Favresse, J.; Brauner, J.; Bodart, N.; Vigneron, A.; Roisin, S.; Melchionda, S.; Douxfils, J.; Ocmant, A., An original multiplex method to assess five different SARS-CoV-2 antibodies. Clin Chem Lab Med 2020, 59, 971-978.
4. Caceres-Martell, Y.; Fernandez-Soto, D.; Campos-Silva, C.; Garcia-Cuesta, E. M.; Casasnovas, J. M.; Navas-Herrera, D.; Beneitez-Martinez, A.; Martinez-Fleta, P.; Alfranca, A.; Sanchez-Madrid, F.; Escudero-Lopez, G.; Vilches, C.; Jara-Acevedo, R.; Reyburn, H. T.; Rodriguez-Frade, J. M.; Vales-Gomez, M., Bead-assisted SARS-CoV-2 multi-antigen serological test allows effective identification of patients. medRyiv 2021, 2021.04.08.21254348.
5. Deshpande, A.; White, P. S., DNA Microarrays and Genetic Testing. In Molecular Diagnostics, 2017; pp 327-337.
6. Heiss, K.; Heidepriem, J.; Fischer, N.; Weber, L. K.; Dahlke, C.; Jaenisch, T.; Loeffler, F. F., Rapid Response to Pandemic Threats: Immunogenic Epitope Detection of Pandemic Pathogens for Diagnostics and Vaccine Development Using Peptide Microarrays. Journal of Proteome Research 2020, 19, 4339-4354.
7. Syu, G. D.; Dunn, J.; Zhu, H., Developments and Applications of Functional Protein Microarrays. Mol Cell Proteomics 2020, 19, 916-927.
8. Brittain, W. J.; Brandsetter, T.; Prucker, O.; RUhe, J., The Surface Science of Microarray Generation—A Critical Inventory. ACS applied materials & interfaces 2019, 11, 39397-39409.
9. Parsa, S. F.; Vafajoo, A.; Rostami, A.; Salarian, R.; Rabiee, M.; Rabiee, N.; Rabiee, G.; Tahriri, M.; Yadegari, A.; Vashaee, D.; Tayebi, L.; Hamblin, M. R., Early diagnosis of disease using microbead array technology: A review. Anal. Chim. Acta 2018, 1032, 1-17.
10. Qu, X.; Bian, F.; Guo, Q.; Ge, Q.; Sun, Q.; Huang, X., Ligation-Rolling Circle Amplification on Quantum Dot-Encoded Microbeads for Detection of Multiplex G-Quadruplex-Forming Sequences. Analytical Chemistry 2018, 90, 12051-12058.
11. Qu, X.; Jin, H.; Liu, Y.; Sun, Q., Strand Displacement Amplification Reaction on Quantum Dot-Encoded Silica Bead for Visual Detection of Multiplex MicroRNAs. Analytical Chemistry 2018, 90 (5), 3482-3489.
12. Kotagiri, N.; Li, Z.; Xu, X.; Mondal, S.; Nehorai, A.; Achilefu, S., Antibody quantum dot conjugates developed via copper-free click chemistry for rapid analysis of biological samples using a microfluidic microsphere array system. Bioconjug Chem 2014, 25, 1272-1281.
13. Ke, Y.; Lindsay, S.; Chang, Y.; Liu, Y.; Yan, H., Self-Assembled Water-Soluble Nucleic Acid Probe Tiles for Label-Free RNA Hybridization Assays. Science 2008, 319, 180-183.
14. Chaput, J.; Lindsay, S.; Yan, H.; Zhang, P. MODIFIED NUCLEIC ACID NANOARRAYS AND USES THEREFOR. U.S. Pat. No. 8,685,894 B2, 2014.
15. Zhao, S.; Tian, R.; Wu, J.; Liu, S.; Wang, Y.; Wen, M.; Shang, Y.; Liu, Q.; Li, Y.; Guo, Y.; Wang, Z.; Wang, T.; Zhao, Y.; Zhao, H.; Cao, H.; Su, Y.; Sun, J.; Jiang, Q.; Ding, B., A DNA origami-based aptamer nanoarray for potent and reversible anticoagulation in hemodialysis. Nature Communications 2021, 12, 358.
16. Dekker, C., Solid-state nanopores. Nature Nanotechnology 2007, 2, 209-215.
17. Feng, Y.; Zhang, Y.; Ying, C.; Wang, D.; Du, C., Nanopore-based fourth-generation DNA sequencing technology. Genomics Proteomics Bioinformatics 2015, 13, 4-16.
18. Shasha, C.; Henley, R. Y.; Stoloff, D. H.; Rynearson, K. D.; Hermann, T.; Wanunu, M., Nanopore-based conformational analysis of a viral RNA drug target. ACS Nano 2014, 8, 6425-30.
19. Zahid, O. K.; Wang, F.; Ruzicka, J. A.; Taylor, E. W.; Hall, A. R., Sequence-Specific Recognition of MicroRNAs and Other Short Nucleic Acids with Solid-State Nanopores. Nano Lett 2016, 16, 2033-2039.
20. Garalde, D. R.; Snell, E. A.; Jachimowicz, D.; Sipos, B.; Lloyd, J. H.; Bruce, M.; Pantic, N.; Admassu, T.; James, P.; Warland, A.; Jordan, M.; Ciccone, J.; Serra, S.; Keenan, J.; Martin, S.; McNeill, L.; Wallace, E. J.; Jayasinghe, L.; Wright, C.; Blasco, J.; Young, S.; Brocklebank, D.; Juul, S.; Clarke, J.; Heron, A. J.; Turner, D. J., Highly parallel direct RNA sequencing on an array of nanopores. Nature methods 2018, 15, 201-206.
21. Dong, Z.; Kennedy, E.; Hokmabadi, M.; Timp, G., Discriminating Residue Substitutions in a Single Protein Molecule Using a Sub-nanopore. ACS Nano 2017, 11, 5440-5452.
22. Waduge, P.; Hu, R.; Bandarkar, P.; Yamazaki, H.; Cressiot, B.; Zhao, Q.; Whitford, P. C.; Wanunu, M., Nanopore-Based Measurements of Protein Size, Fluctuations, and Conformational Changes. ACS Nano 2017, 11, 5706-5716.
23. Fennouri, A.; Przybylski, C.; Pastoriza-Gallego, M.; Bacri, L.; Auvray, L.; Daniel, R., Single Molecule Detection of Glycosaminoglycan Hyaluronic Acid Oligosaccharides and Depolymerization Enzyme Activity Using a Protein Nanopore. ACS nano 2012, 6, 9672-9678.
24. Rivas, F.; Zahid, O. K.; Reesink, H. L.; Peal, B. T.; Nixon, A. J.; DeAngelis, P. L.; Skardal, A.; Rahbar, E.; Hall, A. R., Label-free analysis of physiological hyaluronan size distribution with a solid-state nanopore sensor. Nature Communications 2018, 9, 1037.
25. Karawdeniya, B. I.; Bandara, Y. M. N. D. Y.; Nichols, J. W.; Chevalier, R. B.; Dwyer, J. R., Surveying silicon nitride nanopores for glycomics and heparin quality assurance. Nature Communications 2018, 9, 3278.
26. Im, J.; Lindsay, S.; Wang, X.; Zhang, P., Single Molecule Identification and Quantification of Glycosaminoglycans Using Solid-State Nanopores. ACS Nano 2019, 13, 6308-6318.
27. Bell, N. A. W.; Keyser, U. F., Specific Protein Detection Using Designed DNA Carriers and Nanopores. Journal of the American Chemical Society 2015, 137, 2035-2041.
28. Sze, J. Y. Y.; Ivanov, A. P.; Cass, A. E. G.; Edel, J. B., Single molecule multiplexed nanopore protein screening in human serum using aptamer modified DNA carriers. Nature Communications 2017, 8, 1552.
29. Morin, T. J. Multiplexed Biomarker Quantitation by Nanopore Analysis of Biomaker-Polymer Complexes 2014, WO 2014/210219.
30. Yao, F.; Peng, X.; Su, Z.; Tian, L.; Guo, Y.; Kang, X.-f, Crowding-Induced DNA Translocation through a Protein Nanopore. Analytical Chemistry 2020, 92, 3827-3833.
31. Fang, S.; Bergstrom, D. E., Fluoride-cleavable biotinylation phosphoramidite for 5′-end-labeling and affinity purification of synthetic oligonucleotides. Nucleic acids research 2003, 31, 708-715.

What is claimed is:

1. A nanotrain comprising:
a plurality of single-stranded DNA carriages arranged linearly, each having a unique sequence;
a plurality of complementary DNA sequences each predesigned to be complementary to a single-stranded DNA carriage, and each hybridized with its complementary single-stranded DNA carriage;
a plurality of affinity molecules for capturing one or more targets, wherein each affinity molecule is constructed to attach to a complementary DNA sequence; and
a plurality of flexible linkers connecting every two adjacent single-stranded DNA carriages, wherein each flexible linker at a first end is connected to 5′-end of a first single-stranded DNA carriage and at a second end is connected to 3′-end of a second single-stranded DNA carriage.

2. The nanotrain of claim 1, wherein each single-stranded DNA carriage and each complementary DNA sequence is independently selected from xeno nucleic acid (XNA), peptide nucleic acids (PNA), locked nucleic acid (LNA), and cyclohexenyl nucleic acids (CeNA), wherein preferably each single-stranded DNA carriage and each complementary DNA sequence has a length ranging from 6 to 1000 bases.

3. The nanotrain of claim 1, wherein each complementary DNA sequence is modified to have a functional group that is predesigned to attach an affinity molecule thereto, wherein preferably the functional group is selected from amine, thiol, azide, alkyne, cycloalkyne, or tetrazine.

4. The nanotrain of claim 1, wherein each affinity molecule is independently selected from one or more of nucleic acid, XNA, aptamer, ligand, antibody, antibody's fragment, antigen, nanobody, affibody, protein, and/or carbohydrate.

5. The nanotrain of claim 1, wherein each affinity molecule comprises a microparticle such as a magnetic bead, amine, thiol, azide, alkyne, cycloalkyne, and/or tetrazine.

6. The nanotrain of claim 1, wherein the one or more targets are selected from multiplexed protein markers, single nucleotide polymorphisms (SNPs), DNA and RNA mutations, structural variations of a genome, drug molecules, antibodies, antigens, and glycans.

7. The nanotrain of claim 1, wherein each single-stranded DNA carriage further comprises a protein molecule carrying at least one function orthogonal to those occurring in natural amino acids, such as oxyamine, hydrazine, aldehyde, azide, alkyne, cycloalkyne, alkene, or tetrazine.

8. The nanotrain of claim 1, wherein each single-stranded DNA carriage further comprises a charged polysaccharide that carries functional groups such as thiol, azide, alkyne, cycloalkyne, tetrazine, or oxyamine.

9. The nanotrain of claim 1, wherein each flexible linker comprises a polypeptide having the following structure: 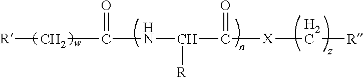
where n=2 to 200; w=1 to 10; z=0 to 10
X=0, NH, or ONH;
R═H, amine, thiol, azide, alkyne, cycloalkyne, tetrazine, carboxylate, hydroxyl, alkyl, alkene, guanidinium, or glycan, selenium;
R′=azide, alkyne, cycloalkyne, tetrazine, or aldehyde;
R″═H, azide, alkyne, cycloalkyne, cyclooctene, tetrazine, or aldehyde.

10. The nanotrain of claim 1, wherein each flexible linker comprises a poly(ethylene glycol) having the following structure: 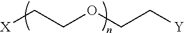
where n=2 to 500;
X=amine, maleimide, vinyl sulfone, cyclooctene, thiol, azide, alkyne, cycloalkyne, tetrazine, oxyamine, carboxylate, or aldehyde;
Y=amine, maleimide, vinyl sulfone, thiol, azide, alkyne, cycloalkyne, tetrazine, oxyamine, carboxylate, or aldehyde.

11. The nanotrain of claim 1, further comprising a neutral tail.

12. The nanotrain of claim 1, further comprising a magnetic bead.

13. A system for single-molecule detection, comprising:
the nanotrain of claim 1,
a nanopore through which the nanotrain translocates, wherein the nanopore is formed by a biological, organic, inorganic, natural or synthetic material and has a pore diameter and thickness in a range of 2 to 1000 nm, preferably 2 to 50 nm; wherein optionally a first pair of electrodes is embedded within the nanopore for measuring current, voltage and/or capacity;
a nanopore device with a cis reservoir and a trans reservoir that are separated by a membrane with the nanopore embedded therein;
a bias voltage that is applied between the cis and trans reservoirs through a second pair of electrodes;
a device for recording a current, voltage or capacity fluctuation caused by the nanotrain translocating through the nanopore; wherein the current, voltage or capacity fluctuation detects one or more targets captured by the affinity molecules; and
software for data analysis that identifies or characterizes the one or more targets.

14. The system of claim 13, wherein the one or more targets are selected from multiplexed protein markers, single nucleotide polymorphisms (SNPs), DNA and RNA mutations, structural variations of a genome, drug molecules, antibodies, antigens, and glycans.

15. The system of claim 13, comprising a plurality of nanotrains, wherein the nanopore device comprises a plurality of nanopores, preferably an array of nanopores, wherein preferably the nanopore device contains 10 to 10⁹ nanopores, preferably 10³ to 10⁷ nanopores, or more preferably 10⁴ to 10⁶ nanopores.

16. A method for single-molecule detection, comprising:
(a) providing the system of claim 13;
(b) mixing the nanotrain with a sample containing one or more targets, thereby forming a loaded nanotrain having the one or more targets captured thereto via the plurality of affinity molecules;
(c) optionally separating the loaded nanotrain from the sample;
(d) placing the loaded nanotrain into the nanopore device, preferably in the cis reservoir;
(e) applying the bias voltage between the cis and trans reservoirs to translocate the loaded nanotrain through the nanopore; and
(f) recording a current, voltage or capacity fluctuation caused by the loaded nanotrain translocating through the nanopore; wherein the current, voltage or capacity fluctuation detects the one or more targets captured by the affinity molecules.

17. The method of claim 16, wherein the method is for high-throughput detection of a plurality of targets, wherein the nanopore device comprises a plurality of nanopores, preferably an array of nanopores, wherein preferably the nanopore device comprises 10 to 10⁹ nanopores, preferably 10³ to 10⁷ nanopores, or more preferably 10⁴ to 10⁶ nanopores.

18. A method of synthesizing a nanotrain, comprising:
(a) providing a plurality of carriages and flexible linkers, wherein each carriage is a single-stranded DNA carriages having a unique sequence, wherein each carriage has orthogonal functional groups attached to its 5′-end and 3′-end, wherein each functional group is independently selected from an amine, maleimide, thiol, vinyl sulfone, azide, alkyne, cycloalkyne, cyclooctene, or oxyamine;
(b) attaching a head carriage via a cleavable linker to a solid support, forming a first end of a nanotrain;
(c) attaching a first flexible linker to the head carriage;
(d) attaching a first carriage to the first flexible linker;
(e) attaching a Nth flexible linker to the (N−1)th carriage, wherein N is an integer >2;
(f) attaching a Nth carriage to the Nth flexible linker;
(g) attaching a (N+1)th flexible linker to the Nth carriage;
(h) repeating steps (e)-(g) until a desired number of carriages are linked by the flexible linkers;
(i) attaching a neutral tail to a second end of the nanotrain; and
(j) cleaving the nanotrain from the solid support at the cleavable linker.
